(19) United States
(12) Patent Application Publication
(10) Pub. No.: US 2006/0153821 A1
(43) Pub. Date: Jul. 13, 2006

(54) Induction of tumor and viral immunity using antigen presenting cell co-culture products and fusion products

(75) Inventors: Louis D. Falo JR., Wexford, PA (US); Christina M. Celluzzi, Columbia, MD (US)

Correspondence Address: FOLEY AND LARDNER LLP;SUITE 500, 3000 K STREET NW, WASHINGTON, DC, 20007, US

(73) Assignee: University of Pittsburgh

(21) Appl. No.: 11/089,025

(22) Filed: Mar. 25, 2005

Related U.S. Application Data
(63) Continuation of application No. 09/864,451, filed on May 25, 2001, now abandoned.
Application No. 09/864,451 is a division of application No. 09/030,985, filed on Feb. 26, 1998, now abandoned.
(60) Provisional application No. 60/039,472, filed on Feb. 27, 1997.

(51) Int. Cl. A61K 48/00; C12N 5/08; C12N 15/86

(52) U.S. Cl. 424/93.21; 435/456; 435/366

(57) ABSTRACT

Formulations comprising combinations of APCs and tumor cells and APCs and virally infected cells are disclosed. These formulations generally comprise hybridoma of at least one antigen presenting cell fused to either at least one tumor cell or at least one virally infected cell, or the products of co-cultures of antigen presenting cells and either tumor cells or virally infected cells. Methods for using these formulations in prophylactically and therapeutically treating patients are also disclosed.

### BACKGROUND OF THE INVENTION

1. Field of the Invention

The present invention relates to formulations for prophylactic and therapeutic anti-tumor and anti-viral immunization and methods for using these formulations in treating a patient. More specifically, the present invention relates to a formulation comprising hybridomas of antigen presenting cells fused to tumor cells or virally infected cells; the present invention further relates to formulations of the products of co-cultures of antigen presenting cells and tumor cells or virally infected cells. The use of these formulations as prophylactic and therapeutic agents against tumors and viral infection is also the subject of the present invention.

2. Background Information

T-cells, including cytotoxic T-lymphocytes (CTLs), are a critical component of effective human immune responses to tumors and viral infections. CTL responses are sufficient to protect against tumors and viruses and can eliminate even established cancers in murine tumor models and in humans. CTLs destroy neoplastic cells or virus infected cells through recognition of antigenic peptides presented by MHC Class I molecules on the surface of the effected target cells. These antigenic peptides are degradation products of foreign proteins present in the cytosol of the effected cell, which are processed and presented to CTLs through the endogenous MHC Class I processing pathway. CTLs target tumors through recognition of a ligand consisting of a self MHC Class I molecule and a peptide antigen. The development of CTL-dependent anti-tumor immunization strategies, therefore, typically depends on both the identification of tumor antigens recognized by CTLs and the development of methods for effective antigen delivery.

Although the recognition of a foreign protein in the context of the MHC Class I molecule may be sufficient for the recognition and destruction of effected target cells by CTLs, the induction of antigen-specific CTLs from T-lymphocyte precursors requires additional signals. Specialized antigen-presenting cells (APCs) can provide both the antigen MHC Class I ligand and the accessory signals required in the induction phase of CTL mediated immunity. General properties of APCs include MHC Class I and Class II expression, expression of various adhesion molecules important for APC-lymphocyte interaction, and expression of co-stimulatory molecules such as CD80 and CD86. APCs include, for example, macrophages, B-cells, and dendritic cells, including cutaneous epidermal Langerhans cells, dermal dendritic cells, and dendritic cells resident in lymph nodes and spleen. Dendritic cells (DCs) are believed to be the most potent APCs, and can induce effective CTL-dependent anti-tumor immunity. Procedures are available to obtain significant quantities of dendritic cells from bone marrow or peripheral blood derived precursors.

It is likely that a tumor cell expresses a set of tumor-specific peptide MHC complexes which can be recognized by CTLs. Progressive tumors, however, are generally non-immunogenic, at least in part, because they are incapable of providing co-stimulation.

Guo et al., Science 263:518-520 (1994), disclose tumor vaccines generated by fusion of hepatoma cells with activated B-cells. The fusion of the activated B-cells and the tumor cells produce an immunogen capable of inducing tumor-specific protective tumor immunity. The present invention offers numerous advantages over this technology by providing for the use of dendritic cells (DC) fused to either tumor cells or virally infected cells. Dendritic cells are believed to be the most potent APCs; DCs can be readily obtained from peripheral blood precursors by established protocols, and DCs do not require the manipulations necessary for activation and maintenance of B-cell APC activity.

Mayordomo et al., Nature Med. 1 (12):1297-1302 (1995), disclose in vitro culture of peptide-pulsed dendritic cells that show protection against the associated tumor challenge. The dendritic cells cultured in the presence of GM-CSF+IL-4 and transfected with chicken ovalbumin (OVA) were capable of preventing establishment of an OVA⁺ tumor, but not the untransfected parental melanoma.

Flamand et al., Eur. J. Inununol. 24:605-610 (1994), disclosed in vitro culture of dendritic cells pulsed with a peptide antigen BCL1, and subsequent induction of a T-cell dependent humoral response to the B-cell tumor BCL1. A similar methodology is reported by Celluzzi et al., J. Exp. Med., 183:203-287 (1996). There, MHC I-peptide antigens were pulsed onto dendritic cells; immunized hosts showed protective immunity to a lethal challenge by a tumor transfected with the antigen gene.

Hsu et al., Nature Medicine 2:52-58 (1996) investigated the use of dendritic cells pulsed with tumor-specific idiotype proteins as vaccines.

There is a need for cancer immunization and immunotherapy procedures that stimulate protective and therapeutic immunity to a wide variety of tumor types. A similar need exists for viral immunization and immunotherapy procedures that stimulate protective and therapeutic immunity to a wide variety of viral infections.

### SUMMARY OF THE INVENTION

The present invention has met the above described need by providing formulations derived from antigen presenting cells and tumor cells. One embodiment of this invention provides a formulation comprising one or more hybridomas comprised of an antigen presenting cell fused to a tumor cell. Another embodiment of this invention provides a formulation comprising the products of co-cultures of antigen presenting cells and tumor cells. These antigen presenting cell/tumor cell formulations can induce CD8+ CTLS, and provide both protection against tumor challenge and regression of tumor growth. Thus, the formulations provide prophylactic resistance to tumors of the type represented by the tumor cell being used in the formulation, and also provides a therapeutic treatment for patients suffering from such tumors.

The present invention also provides formulations comprised of antigen presenting cells and virally infected cells. Again, these formulations can be in the form of one or more hybridomas comprised of antigen presenting cells and virally infected cells, or the products of co-cultures of antigen presenting cells and virally infected cells. These formulations protect against the viral infection caused by the virally infected cells used in the formulation, and/or provide therapeutic relief from patients having such viral infections.

Tumor cells and virally infected cells express antigens which can be targeted by CTLs, but the tumor cells and virally infected cells themselves do not stimulate CTL immunity. This is presumably because the tumor cells and viral cells are incapable of providing the antigen or antigens in the appropriate context of co-stimulation. Antigen presenting cells (APC), however, express a variety of co-stimulatory molecules and cytokines. The present invention provides formulations in which APCs are fused to or are in a co-culture with either tumor cells or virally infected cells. The fused cells and/or co-cultured cells are then used to provide a complete array of tumor antigens or viral antigens that can be delivered to the endogenous pathway of APCs from MHC Class I specific presentation and CTL stimulation. Fusion or co-culture of the APCs with the tumor cells or virally infected cells causes the tumor antigens to become more immunogenic by association with the professional APCs. The fusion products and the co-culture products express properties of both the APC and the tumor; these products are capable of priming a CTL response. This results in the destruction of tumor cells that express similar tumor antigens. Similar results are seen when the formulations comprise products using virally infected cells—the methods of the present invention will result in the destruction of the virus.

As will be appreciated by one skilled in the art, therefore, the present invention obviates the need to identify specific antigens that elicit a CTL response by providing a mechanism that delivers tumor or viral antigens into the MHC class I restricted antigen processing pathway of professional APCs. By delivering the entire array of antigens produced by a tumor cell or a virally infected cell to the APCs, a mechanism is provided for broad, polyvalent immunization.

It is therefore an object of the present invention to provide a hybridoma comprised of APCs and either tumor cells or virally infected cells.

It is a further object of the present invention to provide a pharmaceutical composition comprised of at least one hybridoma of APCs and either tumor cells or virally infected cells.

It is another object of the present invention to provide a pharmaceutical composition comprised of the products of a co-culture of APCs and either tumor cells or virally infected cells.

It is a further object of the present invention to provide a method for the prophylactic treatment of a patient with a formulation comprising at least one hybridoma comprised of APCs fused to either tumor cells or virally infected cells.

It is another object of the present invention to provide a method for the prophylactic treatment of a patient using a formulation comprised of the products of co-culture of APCs and either tumor cells or virally infected cells.

It is yet another object of the present invention to provide a therapeutic treatment to a patient using a formulation comprising at least one hybridoma comprised of APCs and either tumor cells or virally infected cells.

It is another object of the present invention to provide a therapeutic treatment to a patient using a formulation comprised of the products of co-culture of APCs and either tumor cells or virally infected cells.

These and other objects of the invention will be more fully understood from the following description of the invention.

### BRIEF DESCRIPTION OF THE DRAWINGS

FIG. 1 illustrates flow labelling patterns showing the efficiency of association between tumor cell components and dendritic cells, as described in Example 3.

FIG. 2 shows that DC-tumor cell derived vaccines protect mice from lethal tumor challenge, as described in Example 5.

FIG. 3 shows that immunization with DC-tumor cell conjugates results in regression of established tumors and long-lasting anti-tumor immunity, as described in Example 6.

### DETAILED DESCRIPTION OF THE INVENTION

The present invention is directed to a formulation comprising a hybridoma having at least one APC fused to at least one tumor cell. These hybridoma have demonstrated efficacy in providing both prophylactic and therapeutic relief to patients at risk for, or suffering from, tumors. The formulations of the present invention, derived from APCs and tumor cells, are potent immunogens capable of inducing CTL-mediated protective antitumor immunity and the regression of established tumors. Formulations using virally infected cells have a similar immunogenic effect on viral immunity and treatment of viral infections. Also within the scope of the present invention, therefore, is a pharmaceutical composition comprising one or more hybridomas in a suitable pharmaceutical carrier, wherein each hybridoma is comprised of at least one APC fused to at least one tumor cell.

The present invention is also directed to a formulation comprising a hybridoma having at least one APC fused to at least one virally infected cell. A pharmaceutical composition comprising one or more hybridomas in a suitable pharmaceutical carrier, wherein each hybridoma is comprised of at least one APC fused to at least one virally infected cell, is also disclosed.

As will be appreciated by those skilled in the art, a hybridoma is a physical combination of at least two different kinds of cells. At least two different hybridomas fall within the scope of the present invention—namely a hybridoma between at least one APC and one tumor cell, and a hybridoma between at least one APC and at least one virally infected cell.

The present invention is further directed to a formulation comprising the products of co-cultures of APCs and tumor cells. Accordingly, another embodiment of the invention provides a pharmaceutical composition comprising the products of co-cultures of APCs and tumor cells in a suitable pharmaceutical carrier.

The present invention is also directed to a formulation comprising the products of co-cultures of APCs and virally infected cells. Another embodiment of the invention, therefore, provides a pharmaceutical composition comprising products of co-cultures of APCs and virally infected cells in a suitable pharmaceutical carrier.

According to one embodiment of the present invention, one or more APCs, preferably dendritic cells, are fused to one or more tumor cells to form hybridomas or fusion products. Any starting ratio of antigen presenting cells to tumor cells can be used. For example, the starting ratio can be anywhere from 1:10 to 10:1, 1:100 to 100:1, or even higher. In a preferred embodiment, this fusion product is made with a starting ratio of dendritic cells to tumor cells of about 6:1; this starting ratio was found to yield a sufficient number of APC/tumor cell hybridomas. Preferably, this ratio includes a higher number of APCs, as a higher number of APCs increases the probability that the tumor cells will become fused to at least one APC. As will be appreciated by one skilled in the art, one or more APCs can become fused to one or more tumor cells. Thus, the formulations of the present invention generally comprise a number of hybridomas that have a range of APC:tumor cell ratios. For example, when starting with an APC:tumor cell ratio of 6:1, the resulting hybridomas could have an APC:tumor cell ratio of any where from about 1:1 to 10:1 or more.

Any type of tumor cells can be used, including but not limited to, melanoma cells such as melanoma B16 cells, 3LL Lewis lung carcinoma cells, sarcomas, prostate carcinoma, breast carcinoma, colon carcinoma and cervical carcinoma.

Similarly, any type of APCs can be used. As used herein, the term “APC” refers to any cell capable of presenting antigens. APCs include, for example, macrophages, B-cells and dendritic cells (DCs). DCs are found throughout the body, and include cutaneous epidermal Langerhans cells, dermal dendritic cells, dendritic cells located in the lymph nodes and spleen, and dendritic cells derived through in vitro culture of precursors. Antigen presenting cells can be obtained from a host by any means known in the art. For example, dendritic cells can be obtained from bone marrow according to the methods of Celluzzi, et al., J. Exp. Med. 183:283-287 (1996). DCs can also be obtained from peripheral blood and skin. Dendritic cells are the preferred APCs for use in the present invention.

According to another embodiment of the present invention, one or more APCs, preferably DCs, are fused to one or more virally infected cells. As with the APC/tumor cell fusion, any starting ratio of APCs to virally infected cells can be used. Preferably, this ratio is sufficient to allow for the fusion of all of the virally infected cells to one or more APC. Any virally infected cells can be used, including but not limited to, cells infected with influenza virus, human immunodeficiency virus (HIV), cytomegalo virus (CMV), human papilloma virus (HPV) and herpes simplex virus (HSV).

The APCs, tumor cells and/or virally infected cells can be obtained from any source or host. In a preferred embodiment, autologous cells are used.

The hybridomas or fusion products of the present invention can be formed by any method known in the art. In a preferred embodiment, the APC-tumor cell or APC-virally infected cell hybridoma is formed by fusing the two types of cells together with polyethylene glycol (PEG). Generally, this method involves combining the two types of cells and centrifuging the cell suspensions to form a pellet. Approximately 1 ml of a 50% PEG solution heated to about 37° C. should be gradually added to the pellet. The pellet/PEG is gradually diluted with PBS while gentle stirring is applied. The fused cells are then washed by centrifugation and the supernatant decanted, to form the fusion product.

The present invention is also directed to a formulation comprising the products of co-cultures of APCs and either tumor cells or virally infected cells. As used herein, “co-culture” refers to a culture of cells in which there is a population of at least two different types of cells, here APCs and either tumor cells or virally infected cells. A co-culture of APCs and either tumor cells or virally infected cells can be prepared by simply culturing the APCs with either the tumor or virally infected cells. The “products of” this co-culture, as that term is used herein, refers to matter resulting from co-culture of the two types of cells and can include, for example, cells that have become fused together, cells that are not fused, and cellular components including but not limited to the cytoplasm and nuclear matter released from the cell upon cell death or rupture or by other processes. Although any method of co-culturing cells known in the art can be used, in a preferred methodology the two cell types are combined and centrifuged to form a pellet. The pellet is then diluted with a culture medium, preferably RPMI or AIM 5, and incubated overnight at about 37° C. in a 5% CO₂ incubator. As with the hybridoma formulation, any ratio of APC to tumor cell/virally infected cell can be used; a ratio of between about 1:100-100:1 is preferred, a ratio of between about 1:10-10:1 is more preferred and the ratio of about 6:1 is most preferred. It will be appreciated by one skilled in the art that the co-culture product can be used in the formulations and methods of the present invention without a selection step.

The various formulations of the present invention can be used to prepare pharmaceutical compositions useful in the treatment of tumors or viral infections. These pharmaceutical compositions can be prepared by any means known in the pharmaceutical art. Generally, the fusion product or co-culture product according to any of the embodiments of the present invention is combined with a suitable pharmaceutical carrier. Any suitable pharmaceutical carrier can be used, as long as compatibility problems do not arise. The preferred carriers are saline and phosphate buffered saline (PBS).

The present invention is further directed to a method for treating a patient comprising administering to said patient an effective amount of a formulation derived from APCs and tumor cells. This formulation can comprise either at least one hybridoma having at least one APC fused to at least one tumor cell, or can comprise the products of the co-culture of APCs and tumor cells. It will be understood that “treating” includes the prophylactic treatment of a patient susceptible to tumors, such as a patient in a high risk group for certain types of cancer; “treating” also includes the therapeutic treatment of a tumor-bearing patient. APCs and tumor cells as described above can be used. It will be further appreciated that the type of tumor cell used will depend on the type of cancer for which the treatment is being administered. For example, if the patient is being treated to provide prophylactic resistance to melanoma, melanoma cells should be used.

The present invention is further directed to a method for treating a patient comprising administering to said patient an effective amount of a formulation derived from APCs and virally infected cells. This formulation can comprise either at least one hybridoma comprised of at least one APC fused to at least one virally infected cell, or can comprise a co-culture of APCs and virally infected cells. Again, “treating” includes both the prophylactic treatment of a patient prior to a viral infection and the therapeutic treatment of patients having a viral infection. Any APCs and virally infected cells as described above can be used. Again, the type of virally infected cell used will vary depending on the viral infection for which treatment is being provided.

As used herein, the term “effective amount” refers to that amount of a formulation comprising one or more APCs and one or more tumor cell or virally infected cell needed to bring about the desired level of prophylactic resistance or therapeutic relief to a patient. The formulation can comprise, for example, at least one hybridoma having at least one APC fused to either at least one tumor cell or at least one virally infected cell, or the products of co-cultures containing a plurality of APCs and either tumor cells or virally infected cells. As will be appreciated by those skilled in the art, the effective amount will differ from patient to patient depending on such variables as whether the use is prophylactic or therapeutic, the size and/or severity of the tumor or tumors, the type and/or severity of the viral infection, the size and weight of the patient, and the like. It is within the skill of one practicing in the art to determine the effective amount for each patient based upon the general dosage guidelines of between about 1×10⁶ cell equivalents and 100×10⁶ cell equivalents per treatment. Higher or lower doses can be used depending on the patient being treated. The number of treatments will again depend on the patient being treated, the illness being treated, the patient's response to treatments, and the like. Again, it is within the skill of the practitioner to determine the appropriate number of treatments.

When using tumor cells, the methods of the present invention result in the induction of tumor specific lytic activity in an immunized mammalian host. That is, prophylactic or therapeutic treatment will be specific for the type of tumor used in the APC-tumor cell hybridoma or co-culture. Such immunization treatment with the fused cells or the co-culture product protects patients from tumor challenge and/or results in regression of established tumors. Similarly, prophylactic or therapeutic treatment will be specific for the type of virus used, and protects patients from viral challenge and/or results in reduction of viral infection.

As used herein, the term “patients” refers to members of the animal kingdom including but not limited to humans.

### EXAMPLES

The following examples are intended to illustrate the invention, and should not be construed as limiting the invention in any way. The mice used in the examples were female C57BL/6 mice, 5-8 weeks old and were obtained from the Jackson Laboratory in Bar Harbor, Me. B16 is a C57BL/6 melanoma (H-2^{b}) obtained from ATCC, Rockville, Md., and 3LL is a lung carcinoma also available from ATCC. Cell lines were maintained in DME containing 10% FCS and antibiotics. Monoclonal antibodies used to deplete cell subsets were prepared from the hybridomas GK 1.5 (anti-CD4, ATCC T1B 207), 2.43 (anti-CD8, ATCC T1B 210), 30-H12 (anti-Thy 1.2, ATCC T1B107), B220 (anti-B cell surface glycoprotein, ATCC T1B 146), and NK1.1, obtained from W. Chambers, University of Pittsburgh School of Medicine.

### Example 1

### Fusion of DCs and Tumor Cells

Dendritic cells were prepared from bone marrow as generally described in Celluzzi et al., J. Exp. Med. 183:283-287 (1996) using GM-CSF as described in the reference. Briefly, bone marrow cells were depleted of lymphocytes and cultured at 5×10⁵ cells/ml in 10% FCS-containing RPMI 1640, obtained from Irvine Scientific, Santa Ana, Calif., with granulocyte macrophage-colony stimulating factor (GM-CSF), in a concentration of 10³ U/ml, obtained from Sigma Chemical Company, St. Louis, Mo. Loosely adherent cells were collected on day 6 for fusion. Between about 50 and 75% of the DCs expressed CD86 (B7.2) and Class II MHC (I-A⁺) antigens, as determined by flow cytometry.

Day 6 DCs were fused with either B16 or 3LL cells at a ratio of 6:1, DC to tumor cells, using polyethylene glycol at 37° C. After washing by centrifugation, fused cells were cultured overnight at 37° C. in RPMI 1640 (10% FCS).

### Example 2

### Preparation of Dendritic Cell and Tumor Cell Co-Cultures

Dendritic cells were prepared according to the methods of Example 1. Day 6 dendritic cells were then used to form a co-culture with either B16 cells or 3LL cells. Each DC/tumor cell co-culture was prepared by placing DCs into test tubes with the respective tumor cells. A pellet of cells was formed by centrifugation. The pellet was then diluted with RPMI (10% FCS) and incubated overnight at about 37° C. in a 5% CO₂ incubator. The ratio of DCs:tumor cells was about 6:1. The products of the co-culture were prepared to further evaluate whether tumor antigens were present in close association with DCs and to evaluate if soluble factors released from the tumors were present on the DCs. DCs, as well as the supernatants from the transwells, were given to mice as discussed below.

### Example 3

### Efficiency of Fusion and Co-Culture

To determine the efficiency of fusion and co-culture, each of the cell types—DCs, B16 and 3LL—was stained with a different lipophilic fluorochrome before fusion and analyzed using flow cytometry. The tumor cells were stained with DiO while the DCs were stained with DiI, both of which were obtained from Molecular Probes, Inc., Eugene, Oreg. After extensive washing, cells were fused or co-cultured and allowed to incubate overnight at 37° C. Harvested cells were then fixed in 2% paraformaldehyde and the forward and side scatter patterns measured on a Becton Dickinson Facstar Plus with Argon/HeNe duel laser, available from Becton Dickinson Immunocytometry Systems, San Jose, Calif.

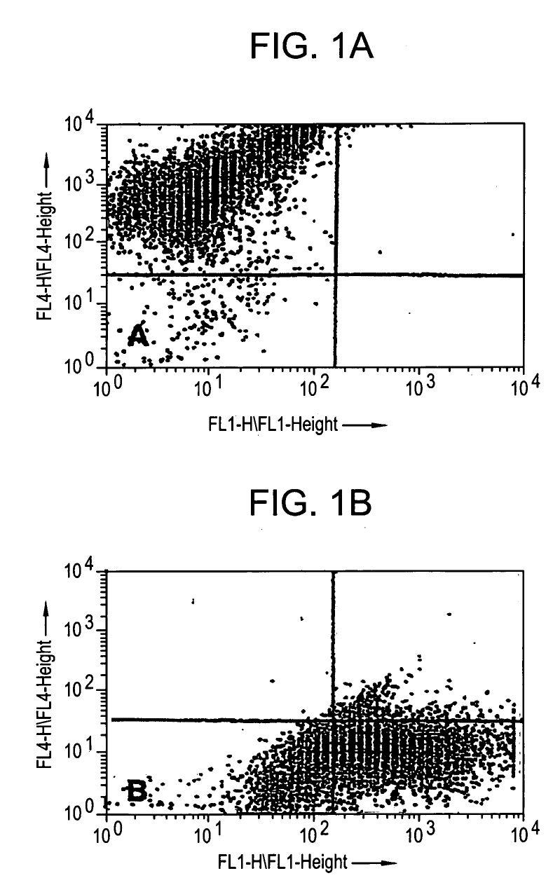

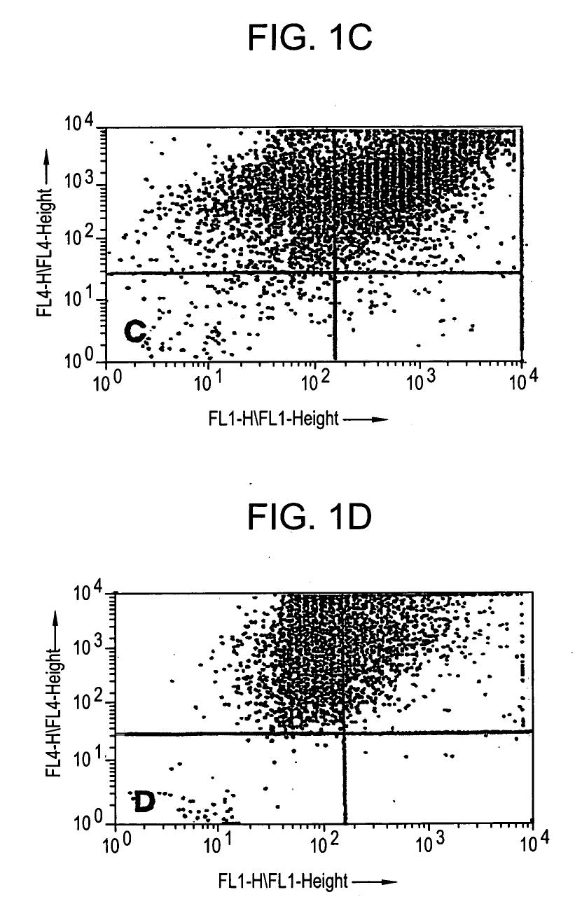

The scatter pattern of each cell type is depicted in FIG. 1. Individual cell staining shows two distinct patterns of DCs (DiI) which shift up (upper left quadrant of FIG. 1A) or B16 tumor cells (DiO) which shift right (lower right quadrant of FIG. 1B) as compared to unstained controls (not shown). When cells were co-cultured (FIG. 1C) or fused (FIG. 1D), the patterns shifted both to the right and up, indicating that those cells were doubly stained. This upper right quadrant was used as the indicator of the “efficiency” of the association of tumor antigens with dendritic cells. Cells found in the upper left or lower right quadrants were regarded as singly stained cells and were not included in the measure. By these standards, the association efficiency was quite high, ranging from about 70% in the co-cultured group to about 53% in the fusion group of the total gated cells.

### Example 4

### Tumor Specificity When Immunized with Fused or Co-Cultured Dendritic Cells and Tumor Cells

The ability of the fused dendritic cells and tumor cells to induce CTLs was examined by injection of DCs and tumor cells from various culture conditions in mice. The conditions included injection of: DCs fused or co-cultured with two different tumor cell lines (either the murine melanoma B16 or the 3LL Lewis lung carcinoma); tumor cells alone; and DCs alone. PBS was used as a control. The fused cells and the products of co-cultured cells were prepared according to the methods of Examples 1 and 2. All of the experimental cell ratios (6 part DCs to 1 part tumor cell) were held constant, and in the cases where tumor cells or DCs alone were injected, amounts were adjusted to include the same number of cell equivalents as were injected from the fusion or co-culture product inoculations.

Groups of mice were immunized with PBS, identical numbers of DCs alone, tumor cells alone, products of DC-tumor cell fusion, or mock fusions (i.e., co-cultures). Some groups of mice were immunized with cells obtained from unfused co-cultures of DCs and tumor cells which had been cultured in separate chambers of transwell plates (transwell cultures) or with identical combinations of DCs and tumor cells injected together without prior co-culture (co-injection). All cellular vaccines were irradiated before subcutaneous injection. Splenocytes were harvested from mice 7 days later and restimulated in vitro with the tumor cell line used to immunize (B16 or 3LL). Lytic activity of effector cells was determined by assay of ⁵¹Cr release from labeled target cells co-incubated with effector cells at 25:1 and 50:1 effector:target ratios. To determine the phenotype of effector cells, splenocytes from mice were depleted of CD4⁺, CD8⁺, NK⁺ or Thy 1.2⁺ cells (where indicated) by incubation with monoclonal antibodies against these markers plus complement and similarly evaluated at 25:1 effector:target ratios for lytic activity. In all cases, <5% lysis was observed against control EL4 tumor targets not used in the immunizations (not shown).

To prepare effector cells, splenocytes (30×10⁶), harvested from mice 7 days after the last immunization (see below), were restimulated by co-culture with irradiated B16 or 3LL cells (7.5×10⁶, 20,000 rad) for 5 days. After this time, cytotoxicity assays were performed as described by Rock et al., The Journal of Immunology, 145:804-811 (1990). Briefly, target cells were labeled by incubation in RPMI with ⁵¹Cr (100 μCi; NEN, Boston, Mass.) for 18 h at 37° C., washed, then co-cultured at 2×10⁴ target cells/well for 4 h at 37° C. in 96-well round-bottom plates (200 μl/well) with effector cells at the ratios given in Table 1. In some cases, effector cells were depleted of CD4⁺, CD8⁺, NK⁺ or Thy 1.2⁺ cells by incubation with monoclonal antibodies against these markers plus complement. 100 μl of supernatants from triplicate co-cultures was collected and counted. Data points are expressed as the mean percent specific release of ⁵¹Cr from target cells and were calculated as described by Rock et al., The Journal of Immunology, 145:804-811 (1990). The standard error of the mean of triplicate cultures was not greater than 5%.

As detailed above, syngeneic naive mice received a single subcutaneous injection of irradiated DC-tumor cell fusion products without adjuvant. Seven days after immunization, spleen cells were restimulated in vitro and then assayed for lytic activity against tumor targets. As controls, groups of mice were immunized with either PBS alone, or equivalent numbers of irradiated DCs or tumor cells alone. In addition, some groups of mice were immunized with irradiated products of co-cultured cells from mock fusions. Splenocytes from mice immunized with tumor-DC fusions demonstrated tumor specific lytic activity. Effector cells from mice immunized with B16-DC fusion lysed B16 targets, but not the irrelevant tumor EL4 (Table 1). Similarly, splenocytes from mice immunized with 3LL-DC fusions demonstrated specific lytic activity against 3LL (Table 1). Importantly, effector splenocytes from mice immunized with products from tumor-DC co-cultures which had not undergone fusion demonstrated similar CTL-mediated tumor-specific lytic activity (Table 1). Depletion of CD8⁺ or Thy 1.2⁺ but not CD4⁺ cell subsets from effector populations using monoclonal antibodies eliminated lytic activity, demonstrating that lysis depended on Thy 1.2⁺ CD8⁺ T cell subsets characteristic of MH class I restricted CTL effector cells (Table 1). In contrast to immunizations with DC-tumor cell fusions or co-culture products, identical numbers of DCs and tumor cells which had not been co-cultured but were injected together at a 6:1 ratio did not induce tumor-specific lytic activity (Table 1, co-injection groups), suggesting that in vitro co-incubation of DCs with tumor cells was necessary for immunogenicity. Furthermore, injection of DCs alone that had been co-cultured with tumor cells at identical ratios and cell densities, but separated by a porous membrane barrier to prevent direct DC-tumor cell contact, did not induce lytic activity (Table 1), suggesting that tumor-specific immunity was not mediated by the transfer of soluble factors from tumor cells to DCs in these in vitro co-cultures.

As described above, where lysis was observed, it was tumor specific. That is, splenocytes from mice immunized with co-cultures or fusion products of DCs and B16 lysed B16 targets while splenocytes from mice immunized with co-cultures or fusion products of DCs and 3LL lysed 3LL targets. Irrelevant targets were not lysed (data not shown). Furthermore, lysis was CD8-T cell mediated as splenocytes depleted for CD8 or Thy 1.2 did not lyse tumor-specific targets.

TABLE 1
A. Non-depleted splenocytes

% specific lysis
(Effector: Target)

| | B16 | Target | 3LL | Target |
|---|---|---|---|---|
| Immunization | 25:1 | 50:1 | 25:1 | 50:1 |
| PBS | 0 | 0 | — | — |
| DCs alone | 0 | 0 | — | — |
| B16 alone | 0 | 0 | — | — |
| B16-DC fusion products | 45 | 100 | — | — |
| B16-DC co-culture products | 65 | 96 | — | — |
| B16-DC co-injection | 0 | 0 | — | — |
| B16-DC transwell cultures | 0 | 0 | — | — |
| PBS | — | — | 0 | 0 |
| DCs alone | — | — | 0 | 0 |
| 3LL alone | — | — | 0 | 0 |
| 3LL-DC fusion products | — | — | 4 | 23 |
| 3LL-DC co-culture products | — | — | 10 | 37 |
| 3LL-DC co-injection | — | — | 0 | 0 |
| 3LL-DC transwell cultures | — | — | 0 | 0 |

B. Splenocytes depleted of:

| | CD4⁺ | CD8⁺ | NK⁺ | Thy1.2⁺ |
|---|---|---|---|---|
| Immunization | % specific lysis | | | |
| PBS | 0 | 0 | 0 | 0 |
| B16-DC fusion products | 68 | 4 | 51 | 0 |
| B16-DC co-culture products | 56 | 0 | 32 | 0 |

### Example 5

### Immunization with PEG-Fused Bone Marrow Derived Dendritic Cells Plus Tumor Cells

To demonstrate the capacity of DC-tumor conjugates to induce anti-tumor immunity in vivo, groups of naive mice were subcutaneously immunized with irradiated DC-tumor cell conjugates without adjuvant and then challenged 7 days later by intradermal injection of the tumor cells in the flanks bilaterally. Specifically, on day 0, C57BL/6 mice were immunized s.c. in both lower flanks (100 μl/side) with DCs alone (1.7×10⁶ per mouse), tumor cells (B16 or 3LL, 3×10⁵ per mouse) alone, fused DCs and tumor cells (B16 or 3LL, 6:1 (i.e., 1.7×10⁶. DCs:3×10⁵ tumor cells per mouse)), products of mock-fused (i.e., co-cultured) DCs and tumor cells (B116 or 3LL, 6:1), identical numbers of DCs and tumor cells (B16 or 3LL, 6:1) injected together without prior co-culture, identical numbers of DCs and tumor cells co-cultured (B16 or 3LL, 6:1) in transwell plates (Costar, Cambridge, Mass.) to prohibit direct cell contact and then injected, supernatants from the same transwell co-cultures (not shown), or PBS. Cells and cell products were irradiated (20,000 rad) and resuspended in PBS before injection. Seven days later, mice were challenged with tumor cells (B16 or 3LL; 5×10⁴/mouse/200 μl at 100 μl/side) in PBS delivered by intradermal injection to the midflanks bilaterally. Surviving mice becoming moribund were sacrificed according to animal care guidelines of the University of Pittsburgh Medical Center. Survival is recorded as the percentage of surviving animals.

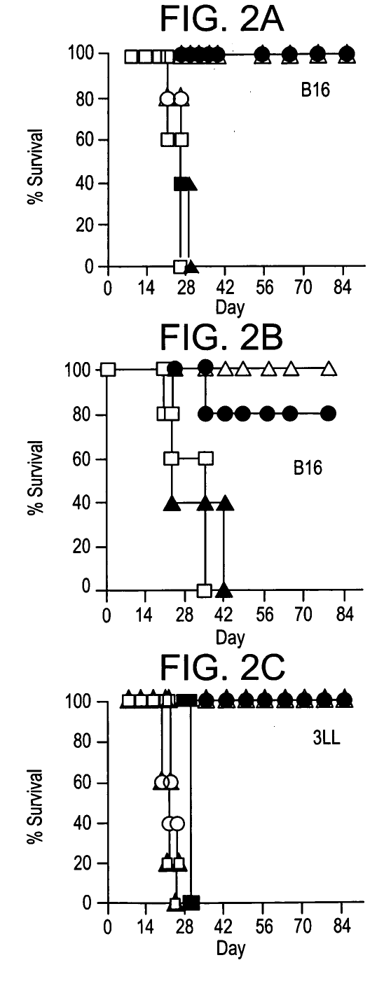

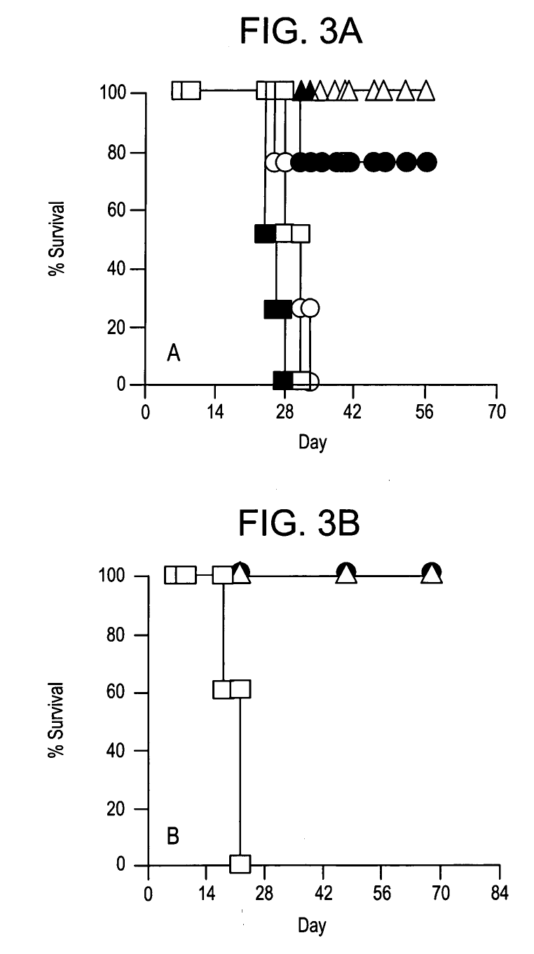

As shown in FIG. 2, C57BL/6 mice were injected s.c. with PBS (open squares), or irradiated vaccines consisting of identical numbers of tumor cells alone (closed squares), DCs alone (open circles), products from DC-tumor fusions (open triangles) or mock fusions (i.e., co-culture, closed circles). Where indicated, mice were immunized with identical numbers of cells derived from DC-tumor cell co-cultures incubated in separate chambers of transwell plates (FIG. 2A, closed triangles) or with DCs and tumor cells injected together without prior co-culture (FIG. 2B, closed triangles). On day 7, mice were challenged intradermally in the midflanks bilaterally with 2.5×10⁴ tumor cells/side and survival was determined as the percentage of surviving animals on a given day for mice immunized and challenged with B16 melanoma (FIGS. 2A and 2B) or 3LL tumor cells (FIG. 2C), n=5 mice/group. Surviving mice had no evidence of tumor when the experiment was terminated. Experiments were repeated twice with similar results.

Mice immunized with irradiated products from DC-B16 fusions or co-cultures were completely protected from lethal challenge with B16 tumor cells (FIG. 2A). Groups of mice injected with PBS, similar numbers of irradiated DCs or tumor cells alone, or irradiated DCs from membrane-separated transwell DC-tumor cell cultures were not protected and uniformly developed lethal tumors (FIG. 2A). Importantly, mice immunized with DCs and tumor cells that were injected together without prior co-culture, were not protected (FIG. 2B). Similarly, immunization with irradiated products from DC-3LL fusions or co-cultures protected mice from challenge with 3LL, while subcutaneous injection of irradiated DCs or tumor cells alone or irradiated DCs from membrane-separated transwell DC-tumor cell co-cultures were ineffective (FIG. 2C).

Taken together, these results demonstrate that immunization with products of DC-tumor cell fusions or co-cultures can induce tumor-specific CTLs and potent protective anti-tumor immunity against two distinct, poorly immunogenic tumors.

### Example 6

### Regression of Pre-Existing 3LL Tumors in Mice

This example demonstrates that the methods of the present invention are effective in treating patients with tumors, as shown by inducing tumor rejection in mice with established tumors. As shown in FIG. 3A, naive C57BL/6 mice were injected intradermally with viable 3LL tumor cells in the midflanks bilaterally (2.5×10⁴ 3LL/side). After tumors were well established (average tumor size 5.9 mm²/mouse, SE +/−0.8), mice were immunized s.c. twice with either PBS (open squares), or identical numbers of irradiated vaccines consisting of 3LL tumor cells alone (closed squares), DCs alone (open circles), products from DC-3LL fusions (open triangles), or mock fusions (i.e., co-culture, closed circles) as described in Example 5. In groups of control mice immunized with either PBS or irradiated DCs or 3LL cells alone, tumors were progressive with no mice surviving beyond day 33 (FIG. 3A). All mice immunized with irradiated DC-3LL fusion products survived for at least 72 days and had no evidence of tumor when the experiment was terminated (FIG. 3A). Immunization with products from co-cultures (mock fusions) prolonged survival in most mice and resulted in complete regression in 75% of the animals treated.

Furthermore, as shown in FIG. 3B, mice demonstrating tumor regression (mice immunized with products of DC-3LL fusion (open triangles) or co-cultures (closed circles)) were rechallenged i.v. 3 months later with 3LL. Non-immunized controls were identically challenged (open squares). Survival is recorded as the percentage of surviving animals on a given day. As shown, tumors did not develop in mice that received fusion products or co-culture products (FIG. 3B), demonstrating that the anti-tumor immunity induced by these vaccines was long lasting. Surviving mice had no evidence of tumor when the experiment was terminated; n=4 mice/group. Experiments were repeated twice with similar results.

Whereas particular embodiments of this invention have been described above for purposes of illustration, it will be evident to those skilled in the art that numerous variations of the details of the present invention may be made without departing from the invention as defined in the appended claims.

1-12. (canceled)

13. A formulation comprising the products of co-cultures of a plurality of first cells and a plurality of second cells; wherein said first cells are antigen presenting cells selected from the group consisting of macrophages, B-cells and dendritic cells, and said second cells are selected from the group consisting of tumor cells and virally infected cells.

14. The formulation of claim 13, wherein said dendritic cells are selected from the group consisting of cutaneous epidermal Langerhans cells, dermal dendritic cells, lymph node dendritic cells, spleen dendritic cells and dendritic cells derived through in vitro culture of precursors.

15. The formulation of claim 13, wherein said tumor cells are selected from the group consisting of melanoma cells, lung carcinoma cells, sarcomas, prostate carcinoma cells, breast carcinoma cells, colon carcinoma cells and cervical carcinoma cells.

16. The formulation of claim 13, wherein said virally infected cells are selected from the group consisting of cells infected with influenza virus, human immunodeficieney virus, cytomegalo virus, human papilloma virus and herpes simplex virus.

17. The formulation of claim 13, wherein said co-culture has a ratio of first cells to second cells of between about 1:100 and 100:1.

18. The formulation of claim 13, wherein said co-culture has a ratio of first cells to second cells of between about 1:10 and 10:1.

19. The formulation of claim 13, wherein said co-culture has a ratio of first cells to second cells of about 6:1.

20. A pharmaceutical composition comprising:
the products of co-cultures of a plurality of first cells and a plurality of second cells; and
a suitable pharmaceutical carrier;
wherein said first cells are antigen presenting cells selected from the group consisting of macrophages, B-cells and dendritic cells, and said second cells are selected from the group consisting of tumor cells and virally infected cells.

21. The pharmaceutical composition of claim 20, wherein the suitable pharmaceutical carrier is selected from the group consisting of saline and phosphate buffered saline.

22. The pharmaceutical composition of claim 20, wherein said co-culture has a ratio of first cells to second cells of between about 1:100 and 100:1.

23. The pharmaceutical composition of claim 20, wherein said co-culture has a ratio of first cells to second cells of between about 1:10 and 10:1.

24. The pharmaceutical composition of claim 20, wherein said co-culture has a ratio of first cells to second cells of about 6:1.

25-30. (canceled)

31. A method for treating a patient comprising:
administering to said patient an effective amount of a formulation comprising the products of co-culture of a plurality of first cells and a plurality of second cells; wherein said first cells are antigen presenting cells selected from the group consisting of macrophages, B-cells and dendritic cells, and said second cells are selected from the group consisting of tumor cells and virally infected cells; and
wherein said administration results in stimulation of a CTL response.

32. The method of claim 31, wherein said dendritic cells are selected from the group consisting of cutaneous epidermal Langerhans cells, dermal dendritic cells, lymph node dendritic cells, spleen dendritic cells and dendritic cells derived through in vitro culture of precursors.

33. The method of claim 31, wherein said tumor cells are selected from the group consisting of melanoma cells, lung carcinoma cells, sarcomas, prostate carcinoma cells, breast carcinoma cells, colon carcinoma cells and cervical carcinoma cells.

34. The method of claim 31, wherein said virally infected cells are selected from the group consisting of cells infected with influenza virus, human immunodeficiency virus, cytomegalo virus, human papilloma virus and herpes simplex virus.

35. The method of claim 31, wherein said formulation further comprises a suitable pharmaceutical carrier.

36. The method of claim 31, wherein said effective amount is between about 1×10⁶ and 100×10⁶ cells.
